(19) United States
(12) Patent Application Publication
(10) Pub. No.: US 2016/0368646 A1
(43) Pub. Date: Dec. 22, 2016

(54) METHOD AND DEVICE FOR PACKAGING ELASTOMER PARTS

(71) Applicant: APTAR STELMI SAS, Villepinte (FR)

(72) Inventors: Raphaël BONA, Saint-Maur-des-Fosses (FR); Ghislain FOURNIER, La Rochelle (FR)

(73) Assignee: APTAR STELMI SAS, Villepinte (FR)

(21) Appl. No.: 15/100,078

(22) PCT Filed: Dec. 12, 2014
(86) PCT No.: PCT/FR2014/053320

§ 371 Date: May 27, 2016

(30) Foreign Application Priority Data
Dec. 16, 2013 (FR) 1362719

(51) Int. Cl. B65B 63/00; B65B 55/10; A61L 2/08; A61J 1/00; B65D 65/38; A61L 2/07; B65B 31/00; B65B 55/16

(52) U.S. Cl. CPC B65B 63/00; B65B 31/00; B65B 55/10; B65B 55/16; A61J 1/00; B65D 65/38; A61L 2/07; A61L 2/081; A61L 2202/23

(57) ABSTRACT

A packaging method for packaging elastomer parts, such as stoppers for pharmaceutical containers. The method includes the steps of washing the parts, packaging the washed parts in a primary bag, drying the parts in the primary bag, packaging the primary bag in a secondary bag that is leaktight and impermeable, packaging the secondary bag in a tertiary bag, and applying a vacuum between the secondary bag and the tertiary bag.

The present invention relates to a packaging method and device for packaging parts made of elastomer, such as stoppers for pharmaceutical containers.

Pharmaceutical containers for containing pharmaceuticals are generally closed, after filling, in leaktight and sterile manner by appropriate stoppers, typically made of elastomer materials. Such stoppers are generally delivered separately from the containers to the user responsible for filling, the stoppers being packaged in appropriate packaging devices. Such ready-to-use stoppers are generally packaged in two bags, namely a primary bag made of polyethylene (PE), in particular made of Tyvek®, that contains the stoppers directly, and a three-layer secondary bag made of polyethylene/polyamide/polyethylene (PE/PA/PE) that is arranged around said primary bag.

Such a packaging system presents drawbacks. Thus, in particular it does not make it possible to guarantee an optimum level of humidity for the stoppers when the bags are opened by the client. Specifically, the insufficient impermeability of the materials that make up said bags does not make it possible to guarantee this optimum level, which causes difficulties while using said stoppers after opening said bags, such as the leaching of moisture from the stoppers to the packaged substance. The same problem may occur for other types of elastomer parts, such as syringe pistons or syringe needle-guards, for example.

Documents EP 0 201 880 and FR 2 756 259 describe prior-art devices.

An object of the present invention is to provide a packaging method and device for packaging elastomer parts that do not have the above-mentioned drawbacks.

In particular, an object of the present invention is to guarantee an optimum level of humidity in the primary bag of the packaging device.

The present invention thus provides a packaging method for packaging elastomer parts, such as stoppers for pharmaceutical containers, the method comprising the following steps:
washing the parts;
packaging the washed parts in a primary bag;
drying the parts in said primary bag;
packaging the primary bag in a secondary bag that is leaktight and impermeable;
packaging said secondary bag in a tertiary bag; and
applying a vacuum between said secondary bag and said tertiary bag.

Advantageously, before said drying step, said primary bag containing said washed parts is subjected to steam sterilization, in particular in an autoclave.

Advantageously, after packaging said secondary bag in said tertiary bag and applying the vacuum, said parts are subjected to gamma ray sterilization.

Advantageously, said primary bag comprises polyethylene, in particular Tyvek®.

Advantageously, said secondary bag includes a layer made of aluminum.

Advantageously, said secondary bag comprises a plurality of layers, in particular a four-layer structure with an inner layer made of polyethylene terephthalate, a layer made of aluminum, a layer made of polyamide, and an outer layer made of polyethylene.

Advantageously, said tertiary bag comprises polyethylene.

The present invention also provides a packaging device for packaging elastomer parts, such as stoppers for pharmaceutical containers, the device being characterized in that it comprises a primary bag that receives said washed parts directly, a secondary bag that is leaktight and impermeable and that receives said primary bag, and a tertiary bag that receives said secondary bag, the gap between said secondary and tertiary bags being subjected to a vacuum.

Advantageously, said primary bag comprises polyethylene, in particular Tyvek®.

Advantageously, said secondary bag includes a layer made of aluminum.

Advantageously, said secondary bag comprises a plurality of layers, in particular a four-layer structure with an inner layer made of polyethylene terephthalate, a layer made of aluminum, a layer made of polyamide, and an outer layer made of polyethylene.

Advantageously, said tertiary bag comprises polyethylene.

These characteristics and advantages of the present invention, and others, appear more clearly from the following detailed description, given by way of non-limiting example, and with reference to the accompanying drawings, in which:

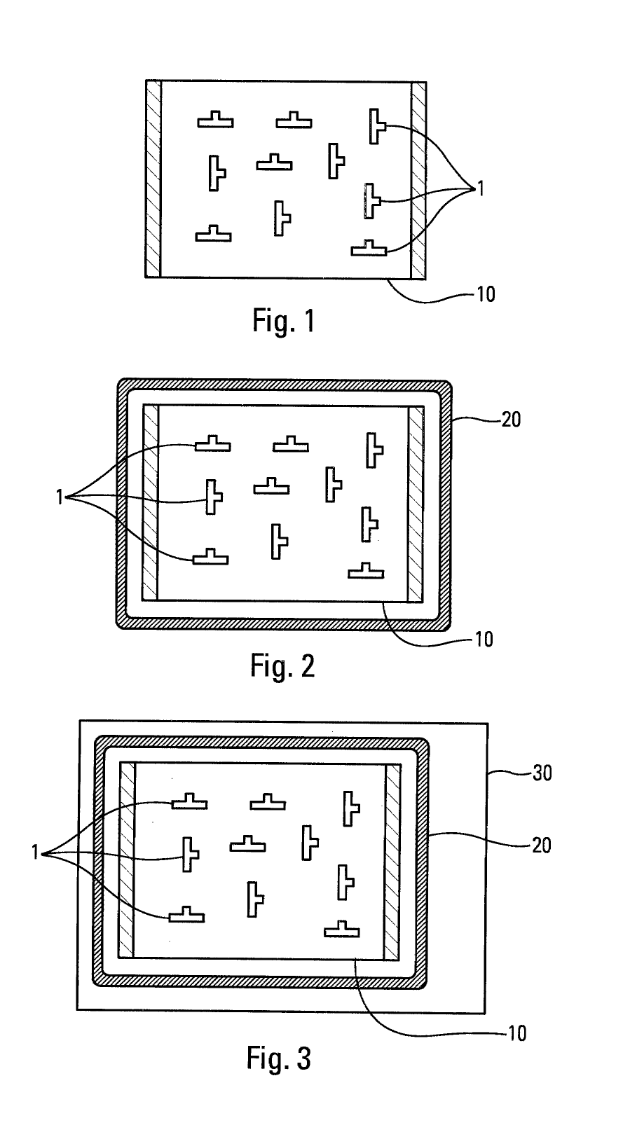

FIG. 1 is a diagrammatic view of a primary bag containing stoppers;

FIG. 2 is a diagrammatic view of a secondary bag that is arranged around the FIG. 1 primary bag;

FIG. 3 is a diagrammatic view of a tertiary bag that is arranged around the FIG. 2 secondary bag; and

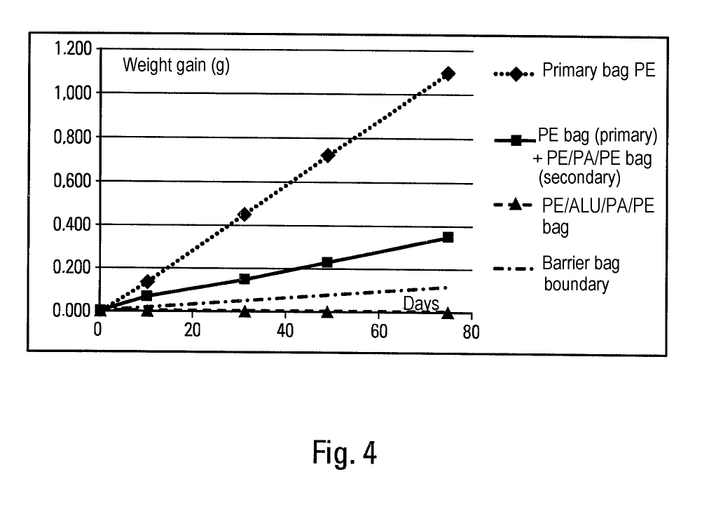

FIG. 4 is a graph showing the permeability test applicable to the secondary bag.

FIGS. 1 to 3 are diagrammatic, and naturally neither the shape of the parts nor the shape of the various bags is representative of the real shapes or to scale. In particular, the parts may be of any number and of any shape, and the bags may also be of any shape and size.

In the invention, elastomer parts 1, such as stoppers for pharmaceutical containers, are packaged in a packaging device that comprises a primary bag 10 that receives said parts directly, a secondary bag 20 that is leaktight and impermeable and that receives said primary bag 10, and a tertiary bag 30 that receives said secondary bag 20. Air is evacuated from between the secondary and tertiary bags 20, 30. This vacuum makes it possible to detect any possible problem of integrity that would appear as a loss of vacuum causing the said secondary and tertiary bags 20, 30 to become separated.

The primary bag 10 advantageously comprises polyethylene, in particular Tyvek®. This material presents a face that is very permeable to steam.

The tertiary bag 30 also advantageously comprises polyethylene. The tertiary bag 30 may be multilayered, e.g. with an inner layer made of polyethylene, a central layer made of polyamide, and an outer layer made of polyethylene. Other materials may also be envisaged providing they make it possible to establish and to maintain a vacuum between the secondary bag 20 and the tertiary bag 30.

The secondary bag 20 advantageously includes aluminum that presents excellent properties of impermeability. By way of example, the secondary bag may comprise a plurality of layers, in particular a four-layer structure with an inner layer made of polyethylene terephthalate, a layer made of aluminum, a layer made of polyamide, and an outer layer made of polyethylene.

In the context of the present invention, the secondary bag 20 is considered to be leaktight and impermeable when it presents satisfactory results in the following permeability test.

### Permeability Test:

This test consisted in measuring the weight gain of a hygroscopic powder (CaCl₂) that was packaged in the material to be tested, and that was exposed in an environment in which humidity and temperature were controlled.

Operating Mode:

The powdered CaCl₂ was pre-dried in an incubator for 72 hours at 150° C.

20 grams (g) of powder were then packaged in a bag made of the material to be tested. The internal dimensions of the bag were 80 millimeters (mm)×80 mm. The bag was hermetically sealed, then placed in a climate chamber that guaranteed a temperature of 25° C. and relative humidity of 60%.

In the context of the present invention, the bag was considered to be leaktight and impermeable when the weight gain recorded after 60 days had not exceeded 0.1 g.

It should be observed on the graph in FIG. 4 that a bag made of polyethylene (PE), such as the primary bag, is very permeable. Likewise, the combination of a primary bag made of PE and a three-layer secondary bag made of polyethylene/polyamide/polyethylene (PE/PA/PE), as used in the prior art, does not make it possible to satisfy the permeability test.

In contrast, a four-layer bag in accordance with an advantageous embodiment of the present invention, is completely leaktight and impermeable, and thus satisfies the permeability test.

Other materials may also be envisaged for the secondary bag 20, providing they make it possible to satisfy the above-mentioned impermeability test.

It should be observed that the thicker the secondary bag, the more leaktight and impermeable it is. Depending on the material selected, it is possible to adapt the thickness of the bag, so as to satisfy the above-mentioned permeability test. In a variant, it is also possible to provide a plurality of secondary bags that are arranged one inside the other, which would achieve the same effect as an increase in the thickness of a single secondary bag.

Two variants are possible for the method of packaging the parts 1.

In a first variant, the parts are initially washed, then packaged in the primary bag. Said primary bag is then subjected to steam sterilization, e.g. in an autoclave. Then to a drying step that makes it possible to achieve a desired level of humidity. After steam sterilization and drying, the primary bag is then packaged in the secondary bag which is in turn packaged in the tertiary bag, with a vacuum being applied between the secondary and tertiary bags.

In a second variant, the parts are initially washed, then packaged in the primary bag. After packaging in the primary bag, the parts are dried so as to achieve a desired level of humidity. After drying, the primary bag is then packaged in the secondary bag which is in turn packaged in the tertiary bag, with a vacuum being applied between the secondary and tertiary bags. Then, the assembly is sterilized by gamma radiation, which can be performed after being boxed and loaded on a pallet.

The invention makes it possible to guarantee the level of humidity in the sterile parts until the bags are opened, regardless of storage conditions and duration of storage.

The invention is described above more particularly with reference to stoppers for pharmaceutical containers, but it could also apply to other types of elastomer parts, such as syringe pistons or syringe needle-guards, for example.

Although the present invention is described above with reference to particular embodiments, naturally the present invention is not limited by those embodiments, and, on the contrary, any useful modification could be applied thereto by the person skilled in the art, without going beyond the ambit of the present invention, as defined by the accompanying claims.

1. A packaging method for packaging elastomer parts (1), such as stoppers for pharmaceutical containers, the method being characterized in that it comprises the following steps:
washing the parts;
packaging the washed parts in a primary bag;
drying the parts in said primary bag;
packaging the primary bag in a secondary bag that is leaktight and impermeable;
packaging said secondary bag in a tertiary bag; and
applying a vacuum between said secondary bag and said tertiary bag.

2. A method according to claim 1, wherein, before said drying step, said primary bag containing said washed parts is subjected to steam sterilization, in particular in an autoclave.

3. A method according to claim 1, wherein after packaging said secondary bag in said tertiary bag and applying the vacuum, said parts are subjected to gamma ray sterilization.

4. A method according to claim 3, wherein said primary bag comprises polyethylene, in particular Tyvek®.

5. A method according to claim 4, wherein said secondary bag includes a layer made of aluminum.

6. A method according to claim 5, wherein said secondary bag comprises a plurality of layers, in particular a four-layer structure with an inner layer made of polyethylene terephthalate, a layer made of aluminum, a layer made of polyamide, and an outer layer made of polyethylene.

7. A method according to claim 6, wherein said tertiary bag comprises polyethylene.

8. A packaging device for packaging elastomer parts, such as stoppers for pharmaceutical containers, the device being characterized in that it comprises a primary bag that receives said washed parts directly, a secondary bag that is leaktight and impermeable and that receives said primary bag, and a tertiary bag that receives said secondary bag, the gap between said secondary and tertiary bags being subjected to a vacuum.

9. A device according to claim 8, wherein said primary bag comprises polyethylene, in particular Tyvek®.

10. A device according to claim 8, wherein said secondary bag includes a layer made of aluminum.

11. A device according to claim 8, wherein said secondary bag comprises a plurality of layers, in particular a four-layer structure with an inner layer made of polyethylene terephthalate, a layer made of aluminum, a layer made of polyamide, and an outer layer made of polyethylene.

12. A device according to claim 8, wherein said tertiary bag comprises polyethylene.
